(19) United States
(12) Patent Application Publication
(10) Pub. No.: US 2006/0287623 A1
(43) Pub. Date: Dec. 21, 2006

(54) Protective cast cover

(76) Inventors: Kent F. Beck, Layton, UT (US); Timothy P. Walker, Park City, UT (US)

Correspondence Address: RANDALL B. BATEMAN;BATEMAN IP LAW GROUP, 8 EAST BROADWAY, SUITE 550, PO BOX 1319, SALT LAKE CITY, UT, 84110, US

(21) Appl. No.: 11/453,270

(22) Filed: Jun. 13, 2006

Related U.S. Application Data
(60) Provisional application No. 60/692,291, filed on Jun. 20, 2005.

(51) Int. Cl. A61F 5/00

(52) U.S. Cl. 602/3

(57) ABSTRACT

A protective cast cover is provided which inhibits damage to other objects, such as furniture and clothing, resulting from collisions between the cast and the object. The cast cover may be made in a skin tone which also makes the cast less obtrusive visually. The covers may be washable, and aid in keeping the cast clean.

### RELATED APPLICATIONS

The present invention claims the benefit of U.S. Provisional Patent Application No. 60/692,291, filed Jun. 20, 2005, which is hereby incorporated herein in its entirety.

### BACKGROUND OF THE INVENTION

1. The Field of the Invention

The present invention relates to medical devices such as casts. More specifically, the present invention relates to a protective cover for use with casts and similar devices to inhibit damage to furniture, clothing, or the like commonly caused by the cast.

2. State of the Art

Casts, splints, or other devices are commonly used to heal broken bones, more severe sprains, etc. As these injuries are not uncommon, many individuals are required to wear a cast or other device at some time. These devices are used for a substantial period of time. Casts, for example, are typically worn for a period of four to six weeks.

Historically, casts have been made of plaster. Recent advances however have changed casting technology. It has been found that a cast formed of fiberglass mesh is better as it allows the skin to breathe better and is more resistant to damage, water, etc.

It is not uncommon for a person wearing such a cast or other such device to inadvertently damage other items such as furniture, clothing, automobiles, etc. The casts are formed from a mesh which is placed around the injured body part, and which needs to be hard so as to prevent accidental movement of a broken bone or injured body part. The cast therefore has many rough or sharp edges and a coarse mesh surface which result from the material used to form the cast. These edges and rough surface are sufficiently sharp and abrasive to damage many common items such as furniture, clothing, etc.

A person with an arm cast may, for example, bump the cast against a wall or furniture, denting, scratching, or gouging the wall or furniture. A person with a cast may also brush the cast against their clothing, tearing or damaging the item of clothing. It is thus not uncommon for a person to damage many items while wearing a cast. The person may even harm themselves or other persons around them, such as by bruising, scraping, or even cutting them as a result of contact between the person and the cast. The damage may even be done while the cast wearer is sleeping.

Another problem with casts is that they get dirty and are difficult to wash. A person wearing a cast must still perform many daily tasks such as eating, working, going to school, etc. Casts quickly get dirty during these everyday activities. The individual wearing the cast is instructed to keep the cast dry, preventing cleaning of the cast. However, the small holes of the mesh readily accept and hold dirt, food, etc.

A further limitation of wearing a cast is the visibility of the cast itself. While a minor inconvenience for a younger person, having a cast may be visually obtrusive for an adult while at work, and especially while at meetings, giving presentations, etc.

It is thus desirable to provide a cast cover. It is desirable to provide a cast cover which protects surrounding objects from being damaged by the cast, which may conceal the cast and make it less noticeable, which protects the cast from becoming dirty, and which may easily be removed and washed.

### SUMMARY OF THE INVENTION

It is an object of the present invention to provide an improved protective cast cover. It is an object to provide a cast cover which protects both the cast and objects contacted by the cast.

According to one aspect of the present invention, a cast cover is provided which protects surrounding items such as furniture or clothing from damage. The cast cover has a surface which is relatively soft and will not damage other items. The cast cover may be padded. The cast cover may typically be made of a sufficiently durable material so as to prevent the cast from cutting through the cover and damaging another item. Preferably, the cast cover is made from a breathable material which provides cushioning between the cast and other objects.

According to another aspect of the present invention, a cast cover is provided which helps to protect the cast from becoming dirty. The use of a material which has a relatively continuous surface, i.e. without substantial holes or openings, will prevent dirt, dust, etc. from contacting the cast. The cover may easily be removed from the cast and washed.

According to another aspect of the present invention, a cast cover is provided which may help make the cast less visible. A cast cover may be provided in colors such as white and various skin tones. The cast will thus be less visible as the cast cover blends in visually with the surrounding skin or clothing.

These and other aspects of the present invention are realized in a protective cast cover as shown and described in the following figures and related description.

### BRIEF DESCRIPTION OF THE DRAWINGS

Various embodiments of the present invention are shown and described in reference to the numbered drawings wherein:

FIG. 1 shows a perspective view of a cast known in the prior art;

FIG. 2 shows a perspective view of a cast cover according to the present invention;

FIG. 3 shows a perspective view of another cast cover according to the present invention;

FIG. 4 shows a perspective view of another cast cover according to the present invention;

FIG. 5 shows a perspective view of yet another cast cover according to the present invention;

FIG. 6 shows a perspective view of another cast cover according to the present invention;

FIG. 7 shows a cross-sectional view of material used to make a cast cover according to the present invention; and

FIG. 8 shows a cross sectional view of a protective cast cover of the present invention placed on a cast.

It will be appreciated that the drawings are illustrative and not limiting of the scope of the invention which is defined by the appended claims. The various embodiments shown accomplish various aspects and objects of the invention. It is appreciated that not all aspects of the present invention are visible in a single drawing, and as such not all elements are visible in a single drawing.

### DETAILED DESCRIPTION

The drawings will now be discussed in reference to the numerals provided therein so as to enable one skilled in the art to practice the present invention. The drawings and descriptions are exemplary of various aspects of the invention and are not intended to narrow the scope of the appended claims.

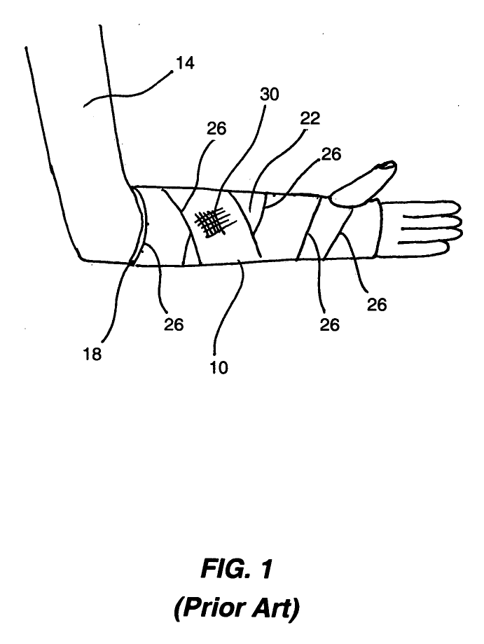

Turning to FIG. 1, a perspective view of a prior art cast is shown. The cast 10 is placed on the arm 14 of an individual. The cast 10 is formed from a padding 18 and multiple layers of a mesh material 22. The mesh material 22 is bonded together and hardened to form a durable cast 10. The process of making a cast 10 results not only in a coarse mesh surface (shown at 30), but in numerous exposed edges 26 of the mesh material.

The exposed edges 26 are sufficiently hard and sharp to scrape or damage wood furniture, scratch surfaces, remove paint, etc. In addition, the open mesh structure 30 acts similarly to sand paper when contacting other objects, causing scratches, removing paint, tearing items, etc.

During any given day a person wearing a cast 10 may damage several items. The person may bump into a wall at home and dent the wall and remove paint. The person may also inadvertently scratch their desk, damage a leather couch or otherwise harm furniture. The person may damage clothing, as simply brushing across a pair of pants or a shirt may tear the clothing. The person may also harm themselves by accidentally contacting their body with the cast or may harm another person by bumping into them with the cast. A person wearing a cast may even harm them self, another person, or furniture while they sleep, as it is common to turn and move while sleeping. It can quickly be seen how a person who is required to wear a cast for four to six weeks may cause a significant amount of damage.

In addition to the damage, the cast 10 may be a nuisance in other regards. Many individuals do not like the appearance of a cast 10. The cast may be visually distracting when working, especially for persons who are required to teach, present, conduct meetings, or otherwise appear before a group of people. Many people feel that a cast 10 is not professional in appearance, or otherwise distracts from their job.

The cast 10 may also become unsightly as it becomes dirty. Casts should be kept dry to prevent skin irritation, fungus growth, etc. and as such are difficult to keep clean. A person required to wear a cast may thus be required to choose between a dark colored cast which will not readily show dirt but which will stand out and be more visually distracting or a light colored cast which may be less visually distracting but which may quickly show dirt.

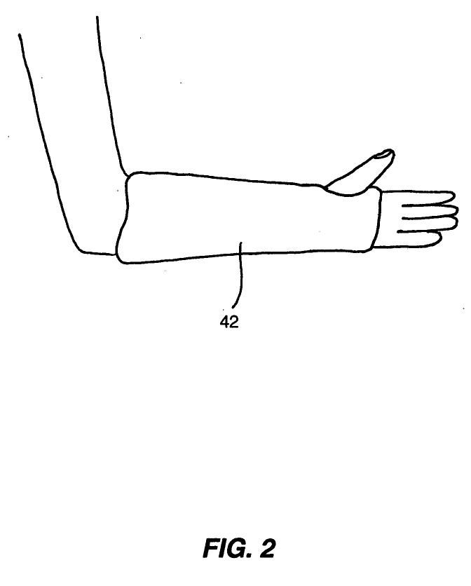

Turning now to FIG. 2, a perspective view of a protective cast cover according to the present invention is shown. The cast cover 42 is typically sized so as to completely cover a cast. The cover 42 is made from a material which will prevent damage to other objects. According to a current embodiment, the cover 42 may be made of a cotton or cotton/polyester blend. According to a more preferred embodiment, the cover 42 may be stretchable so as to accommodate various sizes of casts and thus may have a small amount of an elastic material such as SPANDEX®. The protective cast cover 42 covers the edges and coarse mesh inherent in the cast and inhibits the scratching and damage commonly caused by casts. The cast cover 42 can also provide a small amount of cushioning against impacts.

Several important considerations exist when selecting a material for a cast cover 42. The material should be sufficiently durable so as to not easily tear from contact with the cast or other objects. The material should also be sufficiently soft to protect objects such as furniture. The material should also be convenient for the person wearing the cover 42. Thus, the material should be breathable so as to inhibit the buildup of heat or moisture inside of the cover 42 and cast. The material should also be washable and easily cleaned. Thus, a person may purchase a few cast covers 42 and wear each cover for one day or a few days as is desired, and wash the dirty covers 42 with the laundry.

The material used for the cover 42 may be selected to be thick enough to cushion the cast somewhat, and thereby inhibit denting of other objects. Alternatively, the cast cover may be padded. Several types of fabrics are available which are thus suited. Knit fabrics, such as those used for socks, provide a cushioned fabric which is also durable and stretchable.

The protective cover 42 may also be made in a particular color or colors. As many persons, particularly working professionals, do not desire the appearance of a cast, the protective cover may be made in a variety of skin tones. By making covers 42 in a limited number of colors, most individuals could find a cover which is relatively close to their skin color. Cast covers may thus be made in an off-white color to match pale skin, a color which matches tanned skin, a color which matches darker skin colors, etc. The cast cover 42 would thus make the cast less visually obtrusive. Two-toned colors may also be used so a portion of the cast cover blends in with the skin and another portion blends with clothing, such as a suit coat.

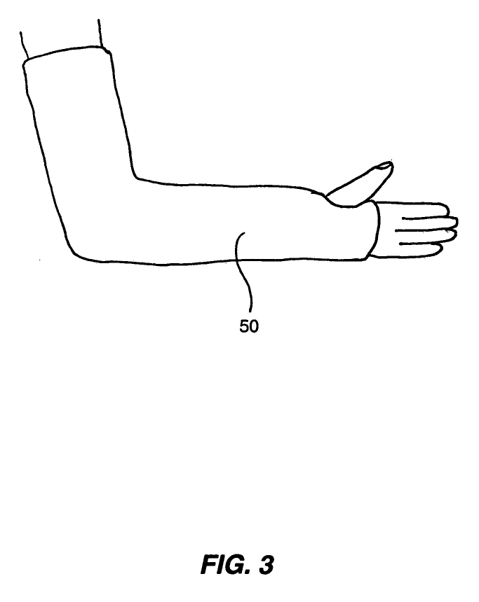

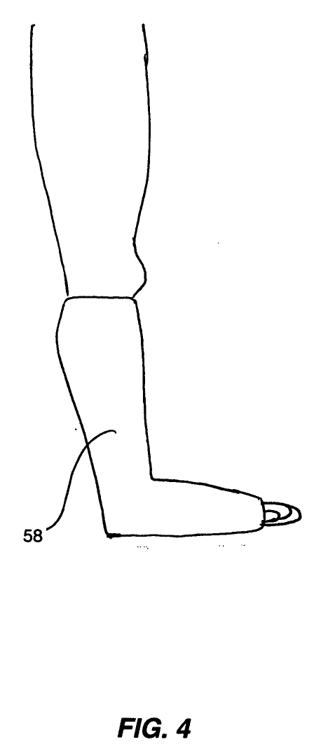

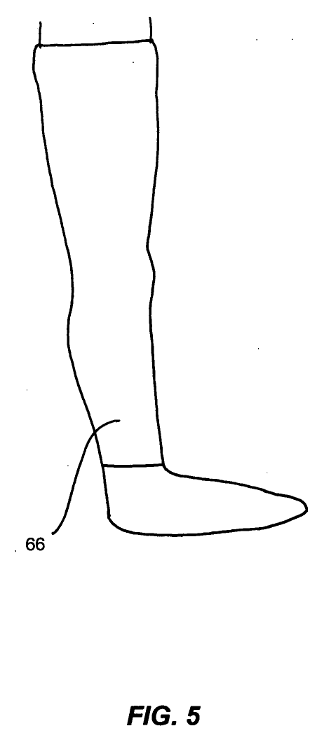

It will be appreciated that a variety of different sizes of protective cast covers may be desirable. The cast cover 42 of FIG. 2 covers a typical forearm cast. FIG. 3 shows a cast cover 50 which covers a whole arm cast. FIG. 4 shows a cast cover 58 designed to cover a lower leg cast, and FIG. 5 shows a cast cover 66 which covers a cast placed over an entire leg. These four sizes of casts are the most common casts used, and are the cast most likely to cause damage to other objects. It is thus preferable to provide cast covers at least for these four types of casts. According to a preferred embodiment of the invention, a cover may be made which is suitable for a particular type of cast, such as a whole arm cast, which will fit both adults and children by using a material which is sufficiently elastic. If desired, however, adult and children sizes may be provided.

It will be appreciated that leg casts can be made to cover the entire foot to thereby not leave the toes exposed. Additionally, the cast cover can be brown, black, etc. around the foot to match the shoe or sock on the person's other foot.

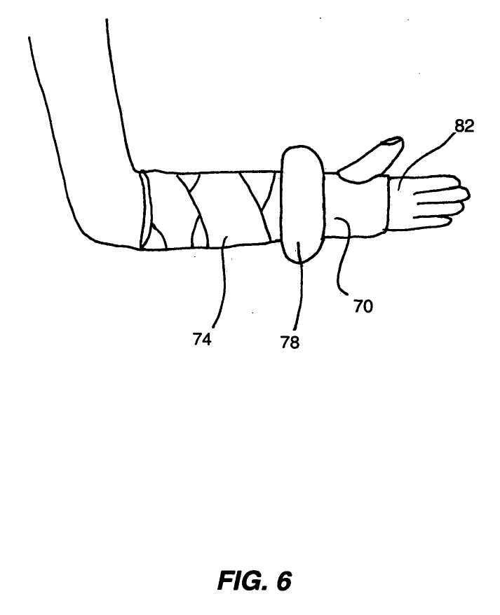

Turning now to FIG. 6, a perspective view of a cast cover according to the present invention is shown. The cast cover 70 is shown partially placed over a forearm cast 74. The cast cover 70 may be provided with a portion thereof formed into a roll 78 such that a person desiring to wear the cover 70 may slide their hand 82 (or foot for a leg cast cover) through the cover 70 and locate the cast cover 70 of the end of the cast 74 as shown. The cast cover 70 may then be unrolled to cover the entire cast 74. A person using the cast cover 70 may also desire to roll the cover as shown to ease placement of the cover 70.

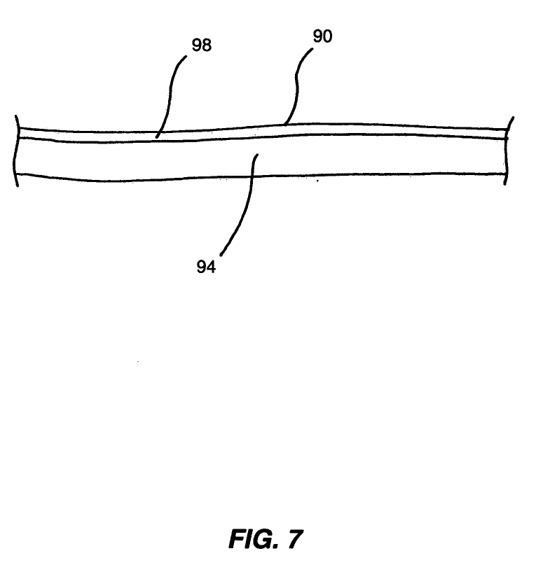

Turning now to FIG. 7, a cross sectional view of cast cover material according to the present invention is shown. The cast cover 90 may be made from a variety of different materials, as has been previously mentioned. It may be desirable to provide a cast cover 90 which has two or more layers forming the cast cover. Thus, the material may comprise a lower layer 94 and an upper layer 98. The lower layer 94 may be a material which provides an amount of padding or cushioning so as to reduce the risk of denting objects which contact the cast. The upper layer 98 may be a material which is resistant to staining, tearing, or other damage to thereby extend the useful life of the cast cover 90 and to help reduce scratching of other objects which contact the cast. Thus, the lower layer 94 may be a loosely knit material or a somewhat thicker material which provides cushioning, and the upper layer may be a tightly knit material which is tear resistant. The layers may be attached by bonding, sewing or other methods known in the textile arts.

Alternatively, the lower layer 94 and upper layer 98 may be part of a single fabric. Some fabrics or methods of forming fabrics produce a material which has both a soft inner layer and a more durable outer layer. Socks or sweatshirts, for example, have both a soft inner layer and an outer layer which is more tightly woven and more durable. It will also be appreciated that the material used to make the cover 90 may have only a single layer. A material may be chosen which is thick enough to provide some padding and protection from tearing, as well as inhibiting damage to other objects which are struck by the cast.

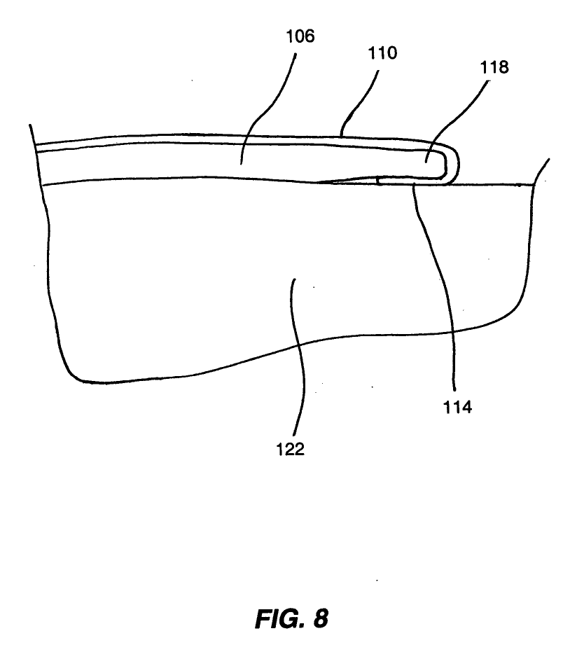

Turning now to FIG. 8, a cross-sectional view of a cast cover placed on a cast is shown. The cast 106 may be an arm cast, leg cast, etc. A protective cast cover 110 has been placed over the cast 106. The edges of casts 118 are particularly abrasive and irritating to the skin, as they contain edges of the fiberglass material used to make the cast. Thus, the casts are often uncomfortable around the edges, and around thumbs, fingers, and toes in particular. An edge 114 of the cast cover 110 has been placed between the edge 118 of the cast 106 and the person's body 122. The cast cover 110 thus helps prevent irritation or abrasion of the body 122.

Thus, the cast cover 110 may be formed so that an edge 114 exists which is suitable for placement between the edge of the cast 118 and the person 122. The cast cover edge 114 may be somewhat thinner that the rest of the cast cover 110, may have a different elasticity than the rest of the cast cover 110, or may be a smoother texture of fabric than the rest of the cast cover 110.

There is thus disclosed an improved protective cast cover. It will be appreciated that numerous changes may be made to the present invention without departing from the scope of the appended claims.

What is claimed is:

1. A protective cast cover comprising:
a sleeve configured for placement over a cast, the sleeve being configured to inhibit the formation of scratches and dents in objects other than the cast caused by collisions between said objects and the cast.

2. The cast cover of claim 1, wherein the cast cover is colored to resemble a skin color.

3. The cast cover of claim 1, wherein the cast cover is biased against the cast.

4. The cast cover of claim 1, wherein an edge of the cast cover is configured for placement between the cast and the person wearing the cast.

5. The cast cover of claim 4, wherein the edge of the cast cover is thinner than the rest of the cast cover.

6. The cast cover of claim 4, wherein the edge of the cast cover has a different texture than the rest of the cast cover.

7. The cast cover of claim 4, wherein the edge of the cast cover has a different elasticity than the rest of the cast cover.

8. The cast cover of claim 1, wherein the cast cover is padded.

9. The cast cover of claim 8, wherein the cast cover comprises a padding layer and a surface layer.

10. The cast cover of claim 1, wherein the cast cover comprises a first color and a second color.

11. The case cover of claim 10, wherein a first end of the cover is the first color and wherein a second end of the cast cover is the second color, and wherein the first color is a skin color.

12. A method of protecting objects from damage caused by a cast, the method comprising comprising:
selecting a cast cover, the cast cover being configured for covering the cast and for preventing contact with the cast; and
placing the cast cover over the cast.

13. The method of claim 12, wherein the method further comprises placing an edge of the cast cover under an edge of the cast.

14. The method of claim 13, wherein the method further comprises selecting a cast cover wherein said edge of the cast cover is of a different thickness than the rest of the cast cover.

15. The method of claim 13, wherein the method further comprises selecting a cast cover wherein said edge of the cast cover is of a different elasticity than the rest of the cast cover.

16. The method of claim 13, wherein the method further comprises selecting a cast cover wherein said edge of the cast cover is of a different surface texture than the rest of the cast cover.

17. The method of claim 12, wherein the method further comprises selecting a cast cover which is stretchable.

18. The method of claim 12, wherein the method further comprises selecting a cast cover which is padded.

19. The method of claim 12, wherein the method further comprises selecting a cast cover which comprises a padding layer and an outer layer of material.

20. The method of claim 12, wherein the method further comprises selecting a cast cover which is skin colored.

21. The method of claim 12, wherein the method further comprises selecting a cast cover which has a first end which is a skin color and a second end which is a different color.

22. The method of claim 12, wherein the method further comprises selecting a cast cover which covers the toes of the wearers foot.
